(19) United States
(12) Patent Application Publication
(10) Pub. No.: US 2003/0212458 A1
(43) Pub. Date: Nov. 13, 2003

(54) Acetabular components providing greater range of motion

(76) Inventors: William H. Harris, Belmont, MA (US); Brian R. Burroughs, Boston, MA (US); Daniel P. Hoeffel, Woodbury, MN (US)

Correspondence Address: HELLER EHRMAN WHITE & MCAULIFFE LLP, 1666 K STREET,NW, SUITE 300, WASHINGTON, DC, 20006, US

(21) Appl. No.: 10/343,413

(22) PCT Filed: Jul. 19, 2001
(86) PCT No.: PCT/US01/22712

(51) Int. Cl. A61F 2/32

(52) U.S. Cl. 623/22.17; 623/22.25

(57) ABSTRACT

The present invention provides monopolar acetabular liners that can hold a femoral head to form a hip replacement prostheses, wherein the liner has a rim that creates and orifice, and wherein the orifice has a diameter that is smaller than the diameter of the femoral head, and wherein there is an outset between the rim of the liner and the center line of the femoral head when the femoral head is disposed therein. Assemblies, complete prosthetic replacements, and methods of replacing also are provided.

This application claims priority to U.S. Serial No. 60/222,044 filed on Jul. 31, 2000, which is incorporated by reference.

The present invention relates to improved acetabular components, such as liners, assemblies and complete replacements, for use in the body. The improved acetabular components can be used, for example, in hip arthroplasties in both a primary and revision setting. The present invention provides marked increases in range of motion (“ROM”) while minimizing the risk of dislocation.

### DESCRIPTION OF THE FIELD

Range of motion is a critical consideration in hip replacements. Acetabular components that yield increased range of motion enhance patient comfort and mobility, and thus overall patient well-being. One approach for achieving increased range of motion is through the use of large diameter femoral heads, such as disclosed in PCT/US99/16070 (the entirety of which is hereby incorporated by reference). Large diameter femoral heads also have greater intrinsic stability than smaller heads in accordance with the principles disclosed in Amstutz et al., Clin. Orthop. 111: 124-30 (1975).

Another concern in hip replacements is dislocation. Dislocation is a major source of morbidity at re-operation after total hip arthroplasty. Dislocation rates have been quoted at 1-10% for primary total hip arthioplasties. Many studies have shown increased rates of dislocation after revision total hip arthroplasty when compared with primary total hip arthroplasty. Currently, the chance of “successful surgical management of a recurrent dislocation” is 70% when a cause for the dislocation can be identified. Dislocation results in significant patient anxiety. In addition, the costs associated with surgical management of dislocation are significant.

A decreased ROM also may contribute to the risk of dislocation. It is postulated that the impingement of the femoral stem on the rim of the insert, due to decreased range of motion of a particular hip replacement design, was the cause of dislocation. Orthopaedic Knlowledge Update, Chapter 38, page 474 (James H. Beatty, M.D., editor, AAOS).

The conventional design of a polyethylene acetabular component for total hip replacement consists of a polyethylene liner for a metal backed shell. The hemisphere of polyethylene has a smaller recess cut into the face of the hemisphere to receive the ball (femoral head) of the femoral component. Such a component might have, for example, a 55 mm outer diameter (OD) and a 32 mm inner diameter (ID). Other variations exist. For example, the recess may be cut deeper into the hemisphere so that the equator of the head is below the face of the hemispherical liner (countersink). Countersink is used to increase the stability of the articulation, because the ball is deeper within the articulation. Countersinking, however, reduces the range of motion (ROM) for the same reason, i.e., the ball is deeper within the polyethylene. In part this restriction in ROM can be offset by creating a chamfer in the polyethylene at the edge of the ID, but in doing so the resistance to dislocation is also compromised.

Thus, current and past designs have utilized various approaches, including countersinks in which the center of rotation of the femoral head is below the level of the flat surface of the liner. These designs, however, were limited in ROM and were at significant risk of dislocation.

Prior designs also have attempted to achieve a comparable ROM by reducing the height of the acetabular component (i.e., the “low profile acetabular components”). While this approach does achieve a similar ROM prior to impingement, it has several limitations. These limitation are discussed herein.

In view of the limitations of the commercialized designs, new approaches are needed that would avoid impingement while at the same time increasing range of motion, which would result in a decreased rate of dislocation for both primary and revision total hip arthroplasties. Such approaches would decrease patient anxiety, eliminate the need for postoperative dislocation precautions, and reduce the number of revisions performed for dislocation and recurrent dislocation resulting in a net cost savings to the healthcare system.

The present invention relates improved acetabular components, such as liners, assemblies and complete replacements, for use in the body. According to the present invention, the recess into which the head is seated is not countersunk, is not colinear with the equator, and potentially has no chamfer. Rather, the recess is brought out (outset) of the hemisphere of polyethylene, as opposed to being countersunk.

Instead of having the femoral head 50% covered by the polyethylene (the radius of the ID of the polyethylene equals the radius of the femoral head), or instead of having the depth of the ID greater than the radius of the femoral head (i.e. the head is countersunk in the polyethylene) we propose to have the radius of the ID and radius of the head identical, but to have the depth of the recess less than the radius of the ID and head. For example, if the head has a 16 mm radius, the recess might be 12 or 14 mm in depth. This means that the femoral head will not be covered to 50% of its area by the polyethylene, it will be outset.

On significant advantage of the present invention's outset design is the increase in the range of motion. This is the result of the decrease in impingement that results from the outset. The femoral neck in this design can travel through a greater ROM before contacting the polyethylene. In fact, if the amount of outset equals the radius of the neck the ROM is 180° and no chamfer is needed.

This present invention is preferable used with a large diameter femoral head, although it is applicable to all head sizes. The use of large diameter femoral heads, for example heads 32 mm and larger, is now feasible because of the development of highly wear resistant, crosslinked ultrahigh molecular weight polyethylene (UHMWPE). The advantage of the larger heads in conjunction with the outset design is as follows:

The outset design has an interrelationship with stability of the articulation. The increase in ROM provides partial protection against dislocation because impingement between the femoral component and the polyethylene is reduced. Impingement is a major contributor to dislocation. On the other hand, with the offset design, the distance that the femoral head must be displaced before it dislocates is reduced by the extent of the outset. This partially increases the risk of dislocation. However, if one compares a 38 mm head with an outset socket to a 32 mm head with or without countersink, the 38 mm head with an outset socket is still placed deeper in the polyethylene. Thus a 38 mm with outset is still intrinsically more stable than the conventional 32 mm head with or without countersink, plus having greater ROM and reduced impingement. In sun therefore, using a 38 mm head with an outset design is preferable to current implant designs. (Compared to a 38 mm head with a conventional recess and ID, it is slightly less stable but has increased ROM.)

### SUMMARY OF THE INVENTION

It is an object of the invention to provide improved prostheses for use in the hip. In accomplishing this and other objects, there is provided in accordance with one aspect of the present invention acetabular liners that can hold femoral heads, including those having large diameters, to form a hip replacement prostheses, wherein the liners have a rim that creates an orifice, and wherein the orifice has a diameter that is smaller than the diameter of the femoral head, and wherein there is an outset between the rim of the liner and the center line of the femoral head when the femoral head is disposed therein. The liners can comprise polyethylene, preferably ultra high molecular weight polyethylene that is cross-linked, preferably by irradiation, more preferably by electron beam irradiation with melting.

In accordance with another aspect of the present invention, there are provided monopolar acetabular assemblies comprising (A) monopolar acetabular liners that can hold a femoral heads, including those having large diameters, to form a hip replacement prostheses; and (B) a metal shell. The liners can comprise polyethylene, preferably ultra high molecular weight polyethylene that is cross-linked, preferably by irradiation. The metal shell can be made of titanium, cobalt chrome, or other suitable materials.

In accordance with still another aspect of the present invention, there are provided hip joint replacement assemblies comprising (A) monopolar acetabular liners that can hold a femoral heads, including those having large diameters, to form a hip replacement prostheses; (B) a metal shell; and (C) a femoral head. The liners can comprise polyethylene, preferably ultra high molecular weight polyethylene that is cross-linked, preferably by irradiation. The femoral head can comprise a femoral stem. One or both of the femoral head and stem can be made of a cobalt chrome alloy, titanium, ceramic, or other suitable materials.

In accordance with yet another aspect of the invention, there are provided methods of replacing a hip in a patient in need thereof, comprising the step of surgically implanting in patients monopolar acetabular liners that can hold a femoral heads including those having large diameters, to form a hip replacement prostheses. The liners can comprise polyethylene, preferably ultra high molecular weight polyethylene that is cross-linked, preferably irradiation. Metal shells, femoral heads and femoral stems also can be implanted according to the invention.

These and other aspects of the present invention will become apparent to the skilled person in view of the description set forth below.

### BRIEF DESCRIPTION OF THE FIGURES

FIG. 1 is a schematic view of a conventional liner wherein the femoral neck is impinging the rim of the liner.

FIG. 2 is a schematic view of a conventional liner having a chamfered edge depicting translation to dislocation of the femoral head out of the socket articulation.

FIG. 3 is a schematic view of an outset liner in functional relation with a femoral head and neck. The polyethylene is a full hemisphere, but the femoral head does not set deeply in to the polyethylene (i.e., it is outset).

FIG. 4 is another view of the liner of FIG. 3 wherein the femoral neck is in contact with rim of the liner. The ROM is 180° before impingement occurs.

### DETAILED DESCRIPTION OF ASPECTS OF THE INVENTION

The present invention increases ROM while decreasing the risk of dislocation of total hip arthroplasty in both the primary and revision setting. In conjunction with large diameter femoral heads, such as disclosed in PCT/US99/16070, the present invention can achieve a range of motion that is greater than is currently available in other systems using 22, 26, 28 and 32 mm heads. The increased range of motion provided by the present invention will afford patients a more normal life style than the currently available systems. The present invention can be surgically implanted in a patient in the same or similar manner as currently employed implants. Thus, the present invention results in an improved quality of life as well as improved patient satisfaction.

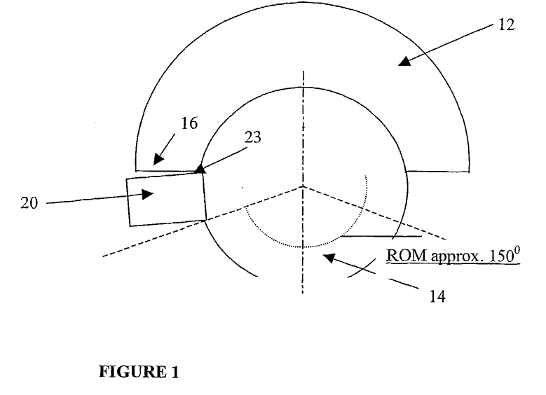

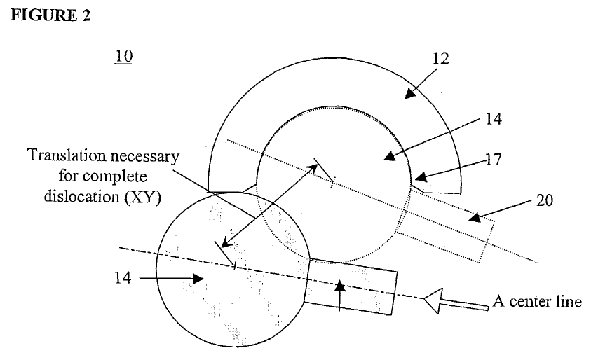

The dislocation phenomenon with conventional acetabular designs is illustrated in FIGS. 1 and 2. Dislocation may be preceded, although not exclusively, by the impingement of the femoral neck against the rim of the acetabular liner. The potential for dislocation also is related to the distance that the femoral head must be displaced in order for the femoral head to escape the acetabular component. Thus femoral head and acetabular component designs which have smaller ROM and smaller are less stable than designs with larger ROM and larger translation to dislocation distances. Outset designs can achieve larger ROM and larger translation to dislocation distances than currently available designs, and thus have a greater intrinsic stability. FIG. 1 represents a conventional system (5) wherein the liner (12) has a rim (16). The femoral head (14) has a neck (20). FIG. 1 shows how the neck (20) impinges (23) the edge of the rim (16), which decreases range of motion, risks break away damage to the rim (16) and can lead to dislocation of the entire head (14) from the liner (12) through a fulcrum like action of the edge of the rim (16) on the neck (20).

FIG. 2 depicts conventional system (10), which is similar to conventional system (5) except that the rim (16) has a chamfered edge (17). The use of the chamfered edge lessens the possibility of damage to the rim (16). This chamfer acts to distribute the load applied by the impinging femoral neck over a greater area hence reducing the stresses within the liner that could otherwise have a damaging effect. A shortcoming of the chamfer is that it serves to promote dislocation. FIG. 2 shows the translation movement need in an X-Y plan needed for total dislocation of the femoral head (14) from the liner (12).

The present invention provides design features which increase the range of motion, decrease the potential for implant impingement, maintain or increase the surface area on the acetabular shell available for bony in-growth, and decrease the significance of component malposition, which often results in impingement/dislocation. These goals are accomplished while maintaining or improving the stability of the hip. These features include, but are not limited to, outset liner design, the potential to eliminate the acetabular chamfer, increased femoral head diameter, and hemispherical shell design.

Components that possess a greater amount of outset will possess greater range of motion of the hip. The benefits of increased range of motion are, (i) decreased potential of component impingement and hence dislocation, (ii) allows a greater degree of freedom at which the acetabular component can be placed without encountering mechanical impingement, (iii) decreased wear and deformity secondary to impingement. The hemispherical design of the outset component provides for (i) decreased load per unit area at the implant cement and/or implant bone interfaces, (ii) decreased torque per unit area at the implant cement and/or implant bone interfaces, (iii) increased press fit of the component into the bony acetabulum, enhancing in-growth of bone into metal backed shells, (iv) increased surface area for bony in-growth using metal backed liners. For a given femoral head size, increasing the amount of outset potentially decreases the stability of the joint by decreasing the amount that the femoral head must be displaced to dislocate. However, this tendency is offset by both (i) the increased ROM before impingement and (ii) the use of larger head sizes.

Increasing the diameter of the femoral head can increase the stability of the joint by increasing the distance that the femoral head must be displaced to dislocate. Therefore, increasing the diameter of the femoral head can offset the potential decrease in joint stability encountered when outset is increased.

The present invention advantageously employs UHMWPE liners, preferably using UHMWPE that is cross-linked, including highly cross-linked UHMWPE. UHMWPE can be cross-linked by a variety of approaches, including those employing cross-linking chemicals and/or irradiation. Preferred approaches for cross-linking employ irradiation, and are taught in PCT/US97/02220, the entirety of which is hereby incorporated by reference.

According to the invention, there is provided a two part acetabular assembly, namely a metal shell for bony in-growth with UHMWPE liner, preferably where the bearing surface of the liner that comes into contact with the femoral head is comprised of crosslinked UHMWPE. Also according to the present invention there is provided an UHMWPE liner designed to be cemented with polymethylmethacralate (PMMA) into the prepared bony acetabular bed.

The liner of the present invention preferably is “monopolar” system. The monopolar acetabular preferably has a one piece design and has a single metal to polyethylene articular surface, and thus preferably is not bipolar or tripolar in design. The monopolar design allows for the use of thicker polyethylene in the acetabular liner. Compared to the bipolar and tripolar constrained/captured designs, the monopolar design decreases the surface area of contact between metal and polyethylene because there is only one metal-polyethylene articulation, rather than two or more found in current and past designs. Moreover, the use of crosslinked UHMWPE will decrease the amount of debris particles generated to articulation. Moreover, different types of heads and liners can be used with the shell, and can be selected by the surgeon during surgery.

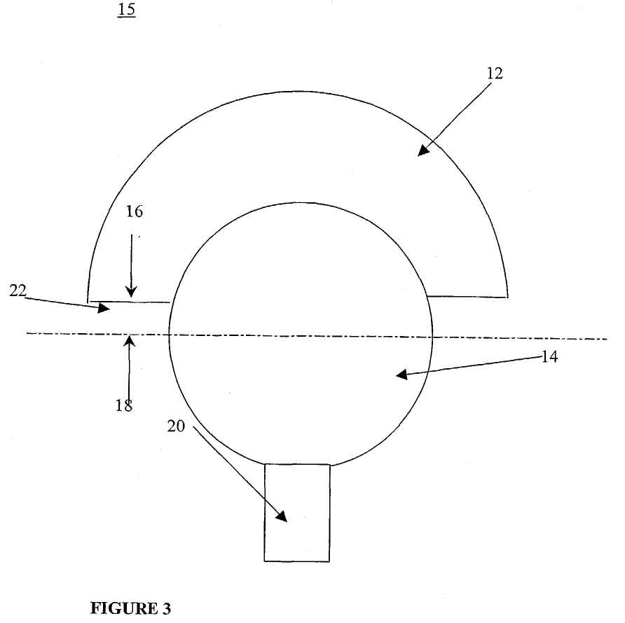

According to one aspect of the invention, less than 50% of the volume of the femoral head is enclosed within the polyethylene liner. FIG. 3 depicts at (15) a liner (12) in functional relation with a femoral head (14) and stem (20). The rim surface (16) of the liner (12) ends before the center line (18), representing the center of rotation, of the femoral head. The perpendicular distance between the plane defined by the rim of the acetabular liner to the center line of the femoral head is referred to as the “outset” (22).

Preferably, the liner rim surface is flat. In other embodiments, for example, modifications such as extended lips, hoods, and long posterior wall liners could be designed with outset characteristics. The inner portions of the rim circumscribe the orifice. A metal shell, not pictured, would be positioned between the liner and the bone at the mount site, and preferably is configured so as to promote in-growth of the bone.

Preferably, the invention is used with a large head diameter (for example, 35 mm or greater, such as 38 mm, 40 mm, 42 mm, 44 mm and any diameter in between or larger) in order to increase the range of motion in comparison with currently available constraint systems.

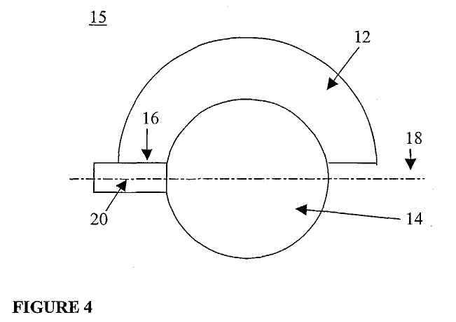

FIG. 4 shows (15) an outset liner (12) wherein the outset is sufficiently great in relation to the radius of the stem (20) such that the stem (20) lies at least substantially flush with most of or the entire surface of the rim (16). This arrangement increases range of motion while at the same time distributes the load applied by the femoral neck over a the entire area of the rim, which reduces the stresses within the liner that could otherwise have a damaging effect. This stress distribution is performed without the need for a chamfered edge, and thus avoid the fulcrum effect of such an edge.

The present invention allows for the use of hemispherical acetabular shells. This in contrast with “low profile” shell. “Low-profile” shells have been designed in the past, and have claimed to have superior ROM in comparison to existing hemispherical designs. The present invention achieves increased ROM though outset and head size. In addition, it has several advantages over the “low-profile” designs, including:

1. A hemispherical design has a greater porous surface for bony in-growth.

2. The press fit of a hemispherical shell within the prepared bony acetabulum is superior to that achieved with the non-hemispherical/low-profile designs.

3. The increased surface area of the liner results in less load and less torque per unit area at the shell bone interface, or the polyethylene-cement and cement bone interfaces when using a cemented acetabular component.

4. The increased surface area of the liner allows for more potential screw locations for those settings in which augmentation with screw fixation is beneficial.

The invention is further demonstrated by the following examples, which do not limit the invention in any manner.

### EXAMPLE 1

Two dimensional modeling was performed to predict ROM. The translational distance to dislocation was defined as X-Y, as depicted in FIG. 2 with regard to a convention chamfered head. The shell modeled was 55 mm outer diameter. The neck of the femoral component was 14 mm in diameter. This diameter was chosen to represent both a worst case scenario and to represent neck sizes currently in use. Smaller neck diameters will produce greater ROM and stability. This diameter is representative of many of currently available femoral stems.

Two dimensional analyses for ROM and translation to dislocation are shown in Tables 1 and 2.

TABLE 1

| Head | Countersink | | Outset | | | | |
|---|---|---|---|---|---|---|---|
| Diameter | −2 mm | −1 mm | 0 mm | 1 mm | 2 mm | 3 mm | 4 mm |
| 32 mm | 114° | 121° | 128° | 135° | 142° | 150° | 157° |
| 38 mm | 125° | 131° | 137° | 143° | 149° | 155° | 161° |

The data of Table 1 show that large heads offer greater range of motion than smaller heads, and that outsets offer greater range of motion than countersinks.

TABLE 2

| | | Distance to Dislocation |
|---|---|---|
| Head Size (mm) | Outset (mm) | (mm) |
| 32 | 0 | 22.6 |
| 38 | 0 | 26.9 |
| 38 | 2 | 25.4 |
| 38 | 4 | 23.9 |

The data of Table 2 demonstrate that 38 mm heads, with and without outsets, have greater translational distances to dislocation than 32 mm heads, which is indicative of stability.

### EXAMPLE 2

A anatomic goniometer testing apparatus was used to measure 3D ROM until impingement. See Krushell et al., J. Arthoplasty 62: 97-101 (1991). This goniometer was used to compare both conventional 32 and 38 mm ID liner designs, and outset liner designs. The polyethylene components tested included: 32 and 38 standard depth, 38 mm ID with 2 mm outset, and 4 mm outset. The heads lengths tested were +4 mm. The acetabular component was placed in a sawbones pelvis in 45° abduction and 20° anteversion. The femoral component was placed in a sawbones femur in 200 anteversion and neutral varus/valgus. The ROM prior to either bony or component impingement was measured in those directions most important to useful range of motion in the activities of daily living and those positions most commonly associated with dislocation. They are flexion, internal rotation at 90° flexion, extension, and extension with external rotation. The location of impingement was also recorded.

Goniometer testing results are shown in Table 3. Increasing the 38 mm head outset from the standard design to 2 mm resulted in an increase in flexion from 143° to 146°. Increasing the outset to 4 mm increased flexion to 149°. Internal rotation at 90° of flexion was increased from 40° to 47°. The 38 mm liner with 4 mm outset has a greater predicted translation to dislocation than the 32 standard design (22.6 mm versus 23.9 mm, see Table 2). In addition, the 38 mm liner with 4 mm outset shows increases of 15° in flexion, 9° in internal rotation at 90° flexion, 15° in extension, and 6° of external rotation at 0° extension in comparison to the standard 32 mm liner.

TABLE 3

| | | | Internal | | External |
|---|---|---|---|---|---|
| Head Size | Outset | | Rotation at | | Rotation at |
| (mm) | (mm) | Flexion | 90° flexion | Extension | 0 Extension |
| 32 | 0* | 134 | 38 | 52 | 50 |
| 38 | 0* | 143 | 40 | 55 | 51 |
| 38 | 2 | 146 | 45 | 70 | 54 |
| 38 | 4 | 149 | 47 | >75 | 56 |

*Liner has a chamfered edge that increases ROM relative to non-chamfered liners.

It is to be understood that the description, specific examples and data, while indicating exemplary embodiments, are given by way of illustration and are not intended to limit the present invention. Various changes and modifications within the present invention will become apparent to the skilled artisan from the discussion, disclosure and data contained herein, and thus are considered part of the invention.

What is claimed is:

1. A monopolar acetabular liner that can hold a femoral head to form a hip replacement prostheses, wherein the liner has a rim that creates an orifice, and wherein there is an outset between the rim of the liner and the center line of the femoral head when the femoral head is disposed therein.

2. The liner according to claim 1, wherein the liner comprises polyethylene.

3. The liner according to claim 2, wherein comprises ultra high molecular weight polyethylene.

4. The liner according to claim 3, wherein ultra high molecular weight polyethylene is cross-linked.

5. The liner according to claim 4, wherein the cross-linking is performed via irradiation.

6. The liner according to claim 1, wherein the femoral head has a large diameter.

7. A monopolar acetabular assembly comprising
(A) a monopolar acetabular liner that encloses a femoral head, wherein the liner has a rim that creates an orifice, and wherein there is an outset between the rim of the liner and the center line of the femoral head when the femoral head is disposed therein; and
(B) a metal shell.

8. The assembly according to claim 7, wherein the metal shell is made of titanium.

9. The assembly according to claim 7, wherein the femoral head has a large diameter.

10. A hip joint replacement assembly comprising
(A) a monopolar acetabular liner that encloses a femoral head, wherein the liner has a rim that creates an orifice, and wherein there is an outset between the rim of the liner and the center line of the femoral head when the femoral head is disposed therein;
(B) a metal shell; and
(C) a femoral head.

11. The replacement according to claim 10, wherein the femoral head comprises a femoral stem.

12. The replacement according to claim 10, wherein the femoral head is made of a cobalt chrome alloy.

13. The replacement according to claim 11, wherein the femoral stem is made of a cobalt chrome alloy.

14. The replacement according to claim 10, wherein the metal shell is made of titanium.

15. The replacement according to claim 10, wherein the femoral head has a large diameter.

16. A method of replacing a hip in a patient in need thereof, comprising the step of surgically implanting in a patient a monopolar acetabular liner that can enclose a femoral head to form a hip replacement prostheses, wherein the liner has a rim that creates an orifice, and wherein there is an outset between the rim of the liner and the center line of the femoral head when the femoral head is disposed therein.

17. The method according to claim 16, wherein a metal shell also is implanted.

18. The method according to claim 16, wherein a femoral head and stem also are implanted.

19. The method according to claim 16, wherein the liner comprises ultra high molecular weight polyethylene.

20. The method according to claim 19, wherein ultra high molecular weight polyethylene is cross-linked.

21. The method according to claim 16, wherein the femoral head has a large diameter.
